(19) United States
(12) Patent Application Publication
(10) Pub. No.: US 2010/0121245 A1
(43) Pub. Date: May 13, 2010

(54) AUXILIARY DEVICE FOR WEARING A TERAPEUTIC ELASTIC STOCKING

(75) Inventor: Roberto Marino, Catania (IT)

Correspondence Address: ROBERTS MLOTKOWSKI SAFRAN & COLE, P.C.;Intellectual Property Department, P.O. Box 10064, MCLEAN, VA, 22102-8064, US

(73) Assignee: Varimed S.R.L., Catania (IT)

(21) Appl. No.: 12/527,645

(22) PCT Filed: Feb. 18, 2008
(86) PCT No.: PCT/IT2008/000101

§ 371 Date: Oct. 16, 2009

(30) Foreign Application Priority Data
Feb. 19, 2007 (IT) RM2007A000089

(51) Int. Cl. A61L 15/00

(52) U.S. Cl. 602/75

(57) ABSTRACT

The invention relates to an auxiliary device for wearing an elastic stocking comprising a first piece of tissue, comprised of slippery material; and a second piece of tissue, comprised of slippery material, coupled along a first portion of its edge with a corresponding first portion of the edge of said first piece of tissue, so as to realise an funnel shaped pocket, closed on its bottom; wherein said first and second pieces of tissue respectively comprise a first and a second guide slot, placed in correspondence of said pocket bottom; and in that it comprises a band having a first end fixed in correspondence of the free portion of said edge of said first piece of tissue and a second end fixed in correspondence of the free portion of said edge of the second piece of tissue, said band passing through said first and second guide slots and having such a length to realise a ring (6′) in correspondence of said slots, said ring being usable for grasping when said device and said elastic stocking are put on, for pulling, folding and sliding each other said first and second pieces of tissue, thus easing extraction of the same device on said stocking.

The present invention relates to an auxiliary device for wearing therapeutic elastic stocking.

More specifically, the invention relates to a device permitting a quick and intuitive wearing of a therapeutic stocking on a limb, aiding users having some motive problems.

As it is well known, after surgical interventions of for different kind of pathologies, elastic stockings are often used, mainly comprised of synthetic fibres, said stocking having high retention properties, suitable to be applied of the concerned limbs, such as a forearm, a foot or a leg.

Said stockings are used in rehabilitative medicine. For example, they are necessary for post-surgical convalescences, such as post-surgical rehabilitation of knee arthroprosthesis.

Furthermore, their use is strongly advised after a deep venous thrombosis, since they can reduce the risk of post-thrombosis complications.

A problem connected with elastic stockings is that they are usually difficult to be put on just because of their high retention capabilities. This problem is particularly important for elderly patients, mainly for their reduced mobility.

Auxiliary devices are available on the market since many time easing the user in wearing said retention elastic stockings on the interested limb.

One kind of device available since many time on the market is comprised of natural fibre slippery tissue comprised of two fabric pieces partially coupled along their edge, so as to realise something similar to “slippers”.

For wearing the elastic stocking the user must first put on the above device. Thanks to the slippery material, it is possible easily putting on the stocking and then removing the same device following its traction by the same user by traction of the top.

Proof the known devices is that often, particularly for an old person, and if the concerned limb is a foot, it is not easy grasping the tip and taking away the device. In fact, contemporaneous sliding between device and user skin during the withdrawal phase often has a remarkable resistance.

In view of the above, it is object of the present invention that of suggesting a device that can be used in an intuitive way and does not require any preparation by the user before being put on.

It is therefore specific object of the present invention an auxiliary device for wearing an elastic stocking comprising a first piece of tissue, comprised of slippery material; and a second piece of tissue, comprised of slippery material, coupled along a first portion of its edge with a corresponding first portion of the edge of said first piece of tissue, so as to realise an funnel shaped pocket, closed on its bottom; characterised in that said first and second pieces of tissue respectively comprise a first and a second guide slot, placed in correspondence of said pocket bottom; and in that it comprises a band having a first end fixed in correspondence of the free portion of said edge of said first piece of tissue and a second end fixed in correspondence of the free portion of said edge of the second piece of tissue, said band passing through said first and second guide slots and having such a length to realise a ring in correspondence of said slots, said ring being usable for grasping when said device and said elastic stocking are put on, for pulling, folding and sliding each other said first and second pieces of tissue, thus easing extraction of the same device on said stocking.

Always according the invention, said first and said second pieces of tissue can be coupled by stitching.

Still according to the invention, said second piece of tissue can have a surface larger with respect to said first piece of tissue.

Furthermore, according to the invention, said first piece of tissue and/or said second piece of tissue can be comprised of cartene e/o nylon e/o tissue and/or synthetic thermoplastic material and/or tetrafluorethylene, polyethylene and/or glass wool.

The present invention will be now described, for illustrative but not limitative purposes, according to its preferred embodiments, with particular reference to the figures of the enclosed drawings, wherein:

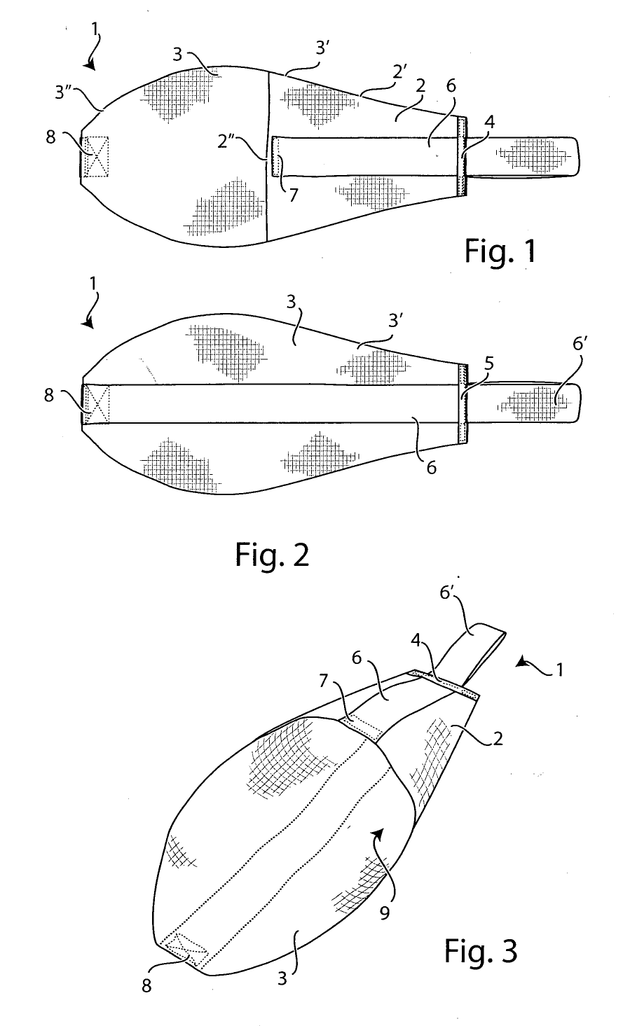

FIG. 1 shows a top plan view of the auxiliary for wearing a therapeutic stocking according to the present invention;

FIG. 2 shows a bottom plan view of the device of FIG. 1;

FIG. 3 shows a perspective view of the device according to FIG. 1;

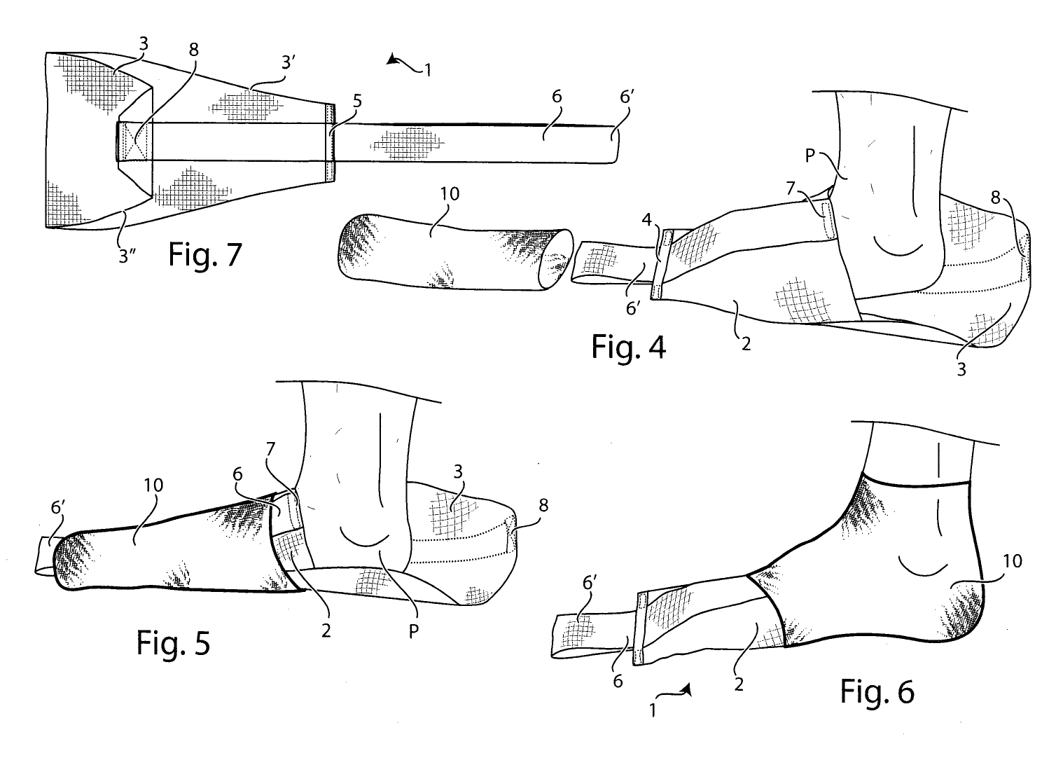

FIG. 4 shows a lateral view of the device according to FIG. 1 put on a user;

FIG. 5 shows a lateral view of device of FIG. 1 put on a user, with an elastic stocking while is being put on the device;

FIG. 6 shows a lateral view of device of FIG. 1 put on a user, with an elastic stocking completely being put on the device; and

FIG. 7 shows a bottom plan view of device according to FIG. 1 with a piece of fabric folded.

Making reference to FIGS. 1, 2 and 3, it is possible observing auxiliary device 1 for wearing a therapeutic stocking according to the invention.

It is noted that device 1 includes a first piece of tissue 2, with its edge comprised of a first 2′ and a second 2″ portion, and a second piece of tissue 3, having its edge comprised of a first 3′ and a second 3″ portion. Said first piece of tissue 2 is stitched along said first portion 2′ of the edge with said second piece of tissue 3 along said first portion 3′ of the edge.

From FIG. 3 it is noted that said first and second pieces of tissues 2, 3 are stitched along relevant portions 2′, 3′ of the edge, realising a kind of pocket 9 or division, within which, in an operative configuration, it can be introduced the limb on which the elastic stocking (not shown in the figure) must be put on.

Said pocket 9 has a closed funnel shape with its bottom stitched.

Said first and second pieces of fabric 2, 3 comprise two guide slots, respectively indicated by reference numbers 4 and 5. said guide slots 4 and 5 are juxtaposed and provided in correspondence of the bottom of the pocket 9, on its lateral edges 2′, 3′.

Device 1 is also provided with a band 6, with a first end 7 stitched on said first piece of fabric 2, in correspondence of the free portion 2″ of its edge, while second end 8 is stitched on said second piece of fabric 3 in correspondence of the free portion 3″ of its edge.

Said band 6 passes through both said guide slots 4, 5. length of said band 6 is such to realise with its exceeding part, a king of handle 6′ in correspondence of said guide slots 4, 5.

FIGS. 4, 5 and 6 show operation of device 1 for easing the work of a user to put on an elastic stocking, said stocking being indicated in the figures by reference number 10. device 1 is shown in its embodiment for putting on an elastic stocking on a foot or on a leg, but the following specification is valid also for putting on elastic stockings 10 on other limbs, suitably varying dimensions of the device 1 and of the elastic stocking 10.

As it can be observed, the user simply introduces the foot P within the pocket 9 realised between said first and second pieces 2, 3 of fabric.

It is clearly noted that:
device 1 does not require a preparation;
introduction of the limb (in this case a foot) within the device 1, and particularly within the pocket 9, is a very natural act.

When the device 1 is put on by a user it is possible easily putting on the elastic stocking 10.

Once put on the elastic stocking 10, being the latter realised in a tubular shape, it permits easily grasping the part of the band 6 comprising a grasping piece of tissue 6′.

Then, for removal of said device 1, that now is between the elastic stocking 10 and the limb, i.e. the foot P or the leg, it is sufficient pulling the grasping piece of tissue 6′ of the band 6.

Said band 6 will make at the same time a traction on both portions 2″, 3″ of the edges of said first and second pieces of tissue 2, 3, that, as it can be observed, are, the first one in correspondence of the feet back, and the second one under the feet P sole.

Both tissue pieces, after traction of the band 6, are folded, thus sliding and making removal of device 1 easier. This effect is particularly evidenced in FIG. 7 for the second piece 3 of fabric, showing that it is folded due to the traction by the band 6.

Device 1 is comprised of low friction synthetic material. Anyway, said device 1 can be comprised of different and materials for each piece of tissue 2, 3, among which nylon, synthetic thermoplastic material film or tissue, tetrafluoroethylene, poli-tetrafluoroethylene, glass wool.

On the basis of the above specification, it can be observed that main advantage of the present invention is that of being usable in a very easy and intuitive way.

A further advantage of the solution according to the present invention is that of being very economic to be manufactures, since it has a very simple structure requiring few materials.

The present invention has been described for illustrative but not limitative purposes, according to its preferred embodiments, but it is to be understood that modifications and/or changes can be introduced by those skilled in the art without departing from the relevant scope as defined in the enclosed claims.

1. An auxiliary device for wearing an elastic stocking comprising a first piece of tissue, comprised of slippery material; and a second piece of tissue, comprised of slippery material, coupled along a first portion of its edge with a corresponding first portion of the edge of said first piece of tissue, so as to realise an funnel shaped pocket, closed on its bottom; wherein said first and second pieces of tissue respectively comprise a first and a second guide slot, placed in correspondence of said pocket bottom; and in that it comprises a band having a first end fixed in correspondence of the free portion of said edge of said first piece of tissue and a second end fixed in correspondence of the free portion of said edge of the second piece of tissue, said band passing through said first and second guide slots and having such a length to realise a ring in correspondence of said slots, said ring being usable for grasping when said device and said elastic stocking are put on, for pulling, folding and sliding each other said first and second pieces of tissue, thus easing extraction of the same device on said stocking.

2. A device according to claim 1, wherein said first and said second pieces of tissue can be coupled by stitching.

3. A device according to claim 1, wherein said second piece of tissue has a surface larger with respect to said first piece of tissue.

4. A device according to claim 1, wherein said first piece of tissue is comprised of cartene e/o nylon e/o tissue and/or synthetic thermoplastic material and/or tetrafluorethylene, polyethylene and/or glass wool.

5. A device according to claim 1, wherein said second piece of tissue is comprised of cartene e/o nylon e/o tissue and/or synthetic thermoplastic material and/or tetrafluorethylene, polyethylene and/or glass wool.

6. A device according to any one of the preceding claims, substantially as illustrated and described.
